(19) United States
(12) Patent Application Publication
(10) Pub. No.: US 2016/0206610 A1
(43) Pub. Date: Jul. 21, 2016

(54) METHODS FOR TREATING AND/OR PREVENTING EMESIS AND/OR NAUSEA INCLUDING ACUTE AND/OR DELAYED NAUSEA AND/OR ACUTE EMESIS AND/OR DELAYED EMESIS

(71) Applicant: Xoc Pharmaceuticals, Inc., LOS GATOS, CA (US)

(72) Inventors: THOMAS ARMER, LOS ALTOS, CA (US); GEOFF MCKINLEY, MOUNTAIN VIEW, CA (US); SCOTT BORLAND, LOS GATOS, CA (US); MIGUEL GUZMAN, CUPERTINO, CA (US)

(21) Appl. No.: 15/001,248

(22) Filed: Jan. 20, 2016

Related U.S. Application Data
(60) Provisional application No. 62/105,212, filed on Jan. 20, 2015.

(51) Int. Cl. A61K 31/473; A61K 9/00

(52) U.S. Cl. CPC A61K 31/473; A61K 9/0043; A61K 9/0075; A61K 9/0078; A61K 9/008

(57) ABSTRACT

Methods for treating and/or preventing nausea and emesis including acute and/or delayed nausea and/or acute and/or delayed emesis in a subject are provided herein. The methods include administering to the subject in need thereof a therapeutically effective amount of a 5HT₃ antagonist or a pharmaceutical composition thereof by nasal inhalation and/or oral inhalation.

This application claims priority under 35 U.S.C. §119 (e) from U.S. Provisional Application Ser. No. 62/105,212 filed Jan. 20, 2015, which is hereby incorporated by reference in its entirety.

### FIELD

Methods for treating and/or preventing nausea and emesis including acute and/or delayed nausea and/or acute and/or delayed emesis in a subject are provided herein. The methods include administering to the subject in need thereof a therapeutically effective amount of a 5HT₃ antagonist or a pharmaceutical composition thereof by nasal inhalation and/or oral inhalation.

### BACKGROUND

Antagonists of the 5HT₃ receptor are known to ameliorate nausea and emesis associated with neoplastic disease, radiation sickness, gastroenteritis, pregnancy, migraine, and the side effects of opioids, cytotoxic drugs, and general anesthetics. Inconsistent dosing as a result of emesis, slow absorption, slow onset and short duration of action diminish the utility of oral administration of 5HT₃ antagonists, despite the convenience of self administration in a non-clinical setting. The rapid onset and extended relief provided by intravenous administration of 5HT₃ antagonists are counterbalanced by burdensome administration which requires costly clinical supervision. The above problems associated with oral and intravenous administration has prevented the development of therapy which effectively treats and prevents acute and/or delayed nausea and/or acute and/or delayed emesis.

Accordingly, what is needed are methods for treating and/or preventing nausea or emesis including acute and/or delayed nausea and/or acute and/or delayed emesis with 5HT₃ antagonists which provide rapid onset and extended relief without inconvenient administration in a clinical setting.

### SUMMARY

The present invention satisfies these and other needs by providing methods, devices and kits for treating nausea and/or emesis, including acute nausea and/or acute emesis in a subject. Generally, the methods include administering one or more 5HT₃ antagonists or pharmaceutical compositions thereof via oral or nasal inhalation. In some embodiments, the 5HT₃ antagonists may be combined with one or more other therapeutic agents, such as dopamine receptor antagonists, antihistamines, long-acting sedative-hypnotic, phenothiazines, cannabinoids, glucocorticosteroids, anticholinergics (e.g. atropine, doxylamine, tiotropium, etc.), NK1 antagonists or combinations thereof. Methods described herein may include administering a therapeutically effective amount of a 5HT₃ antagonist or pharmaceutical composition thereof via oral inhalation and/or nasal inhalation, such that there is rapid onset of action with minimal adverse side effects (e.g., undesirable gastrointestinal, cardiopulmonary or central nervous system effects). In some embodiments, a 5HT₃ antagonist or pharmaceutical composition thereof may be administered using inhalation devices, which include an aerosol spray generating mechanism and a 5HT₃ antagonist or pharmaceutical composition thereof.

In addition to comprising one or more 5HT₃ antagonists, the pharmaceutical compositions described herein may include one or more excipients, such as propellants, carrier media, surfactants, stabilizers, flocculating agents, thickening agents, adhesive agents, absorption enhancers, solvents, dispersants, preservatives, antioxidants, buffering agents, and/or flavoring agents. Depending on the type of inhalation device employed, 5HT₃ antagonists or pharmaceutical composition thereof may be contained within a pressurized canister, blister, capsule, ampoule, spray dispenser, etc. or provided as a solid, which can be scraped, ground, crushed, pulverized, or the like, to form particles.

As described above, oral and/or nasal inhalation of a 5HT₃ antagonist or pharmaceutical composition thereof may be used to treat or prevent nausea and/or emesis, including acute nausea and/or acute emesis in a subject. For example, nausea and/or emesis arising from neoplastic disease, morning sickness during pregnancy, migraine, the side effects of cytological drugs used to treat cancer, opioids and general anesthetics may be treated or prevented by using the methods described herein.

In a first aspect, methods of treating and/or preventing acute and/or delayed nausea and/or acute and/or delayed emesis in a subject are provided. The methods include administering to the subject in need thereof, a therapeutically effective amount of a 5HT₃ antagonist or a pharmaceutical composition thereof by nasal inhalation and/or oral inhalation prior to an event which induces acute and/or delayed nausea and/or acute and/or delayed emesis.

In a second aspect, methods of treating nausea and/or emesis including acute and/or delayed nausea and/or acute and/or delayed emesis in a subject are provided. The methods include administering to the subject in need thereof a therapeutically effective amount of a 5HT₃ antagonist or a pharmaceutical composition thereof, by nasal inhalation and/or oral inhalation.

In a third aspect, methods of preventing nausea and/or emesis including acute and/or delayed nausea and/or acute and/or delayed emesis in a subject are provided. The methods include administering to the subject in need thereof a therapeutically effective amount of a 5HT₃ antagonist or a pharmaceutical composition thereof, by nasal inhalation and/or oral inhalation.

### DETAILED DESCRIPTION

“Hydrates” refers to incorporation of water into to the crystal lattice of a compound described herein, in stoichiometric proportions, resulting in the formation of an adduct. Methods of making hydrates include, but are not limited to, storage in an atmosphere containing water vapor, dosage forms that include water, or routine pharmaceutical processing steps such as, for example, crystallization (i.e., from water or mixed aqueous solvents), lyophilization, wet granulation, aqueous film coating, or spray drying. Hydrates may also be formed, under certain circumstances, from crystalline solvates upon exposure to water vapor, or upon suspension of the anhydrous material in water. Hydrates may also crystallize in more than one form resulting in hydrate polymorphism. (See e.g., (Guillory, K., Chapter 5, pp. 202-205 in Polymorphism in Pharmaceutical Solids, (Brittain, H. ed.), Marcel Dekker, Inc., New York, N.Y., 1999)). The above methods for preparing hydrates are well within the ambit of those of skill in the art, are completely conventional and do not require any experimentation beyond what is typical in the art. Hydrates may be characterized and/or analyzed by methods well known to those of skill in the art such as, for example, single crystal X-ray diffraction, X-ray powder diffraction, polarizing optical microscopy, thermal microscopy, thermogravimetry, differential thermal analysis, differential scanning calorimetry, IR spectroscopy, Raman spectroscopy and NMR spectroscopy. (Brittain, H., Chapter 6, pp. 205-208 in Polymorphism in Pharmaceutical Solids, (Brittain, H. ed.), Marcel Dekker, Inc. New York, (1999)). In addition, many commercial companies routine offer services that include preparation and/or characterization of hydrates such as, for example, HOLODIAG, Pharmaparc II, Voie de l'Innovation, 27 100 Val de Reuil, France (http://www.holodiag.com).

“Preventing” or “prevention” refers to a reduction in risk of acquiring a disease or disorder (i.e., causing at least one of the clinical symptoms of the disease not to develop in a patient that may be exposed to or predisposed to the disease but does not yet experience or display symptoms of the disease). In some embodiments, “preventing” or “prevention” refers to reducing symptoms of the disease by taking a compound in a preventative fashion. The application of a therapeutic for preventing or prevention of a disease of disorder is known as ‘prophylaxis.’ In some embodiments, the compounds provided herein provide superior prophylaxis because of lower long term side effects over long time periods. It should be understood that that prevention and prophylaxis are used interchangeably herein and are thus equivalent.

“Salt” refers to a salt of a compound, which possesses the desired pharmacological activity of the parent compound. Such salts include: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl) benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or (2) salts formed when an acidic proton present in the parent compound is replaced by a metal ion, e.g., an alkali metal ion, an alkaline earth ion, or an aluminum ion; or coordinates with an organic base such as ethanolamine, diethanolamine, triethanolamine, N-methylglucamine and the like. In some embodiments, the salt is pharmaceutically acceptable.

“Solvates” refers to incorporation of solvents into to the crystal lattice of a compound described herein, in stoichiometric proportions, resulting in the formation of an adduct. Methods of making solvates include, but are not limited to, storage in an atmosphere containing a solvent, dosage forms that include the solvent, or routine pharmaceutical processing steps such as, for example, crystallization (i.e., from solvent or mixed solvents) vapor diffusion, etc. Solvates may also be formed, under certain circumstances, from other crystalline solvates or hydrates upon exposure to the solvent or upon suspension material in solvent. Solvates may crystallize in more than one form resulting in solvate polymorphism. (See e.g., (Guillory, K., Chapter 5, pp. 205-208 in Polymorphism in Pharmaceutical Solids, (Brittain, H. ed.), Marcel Dekker, Inc., New York, N.Y., 1999)). The above methods for preparing solvates are well within the ambit of those of skill in the art, are completely conventional do not require any experimentation beyond what is typical in the art. Solvates may be characterized and/or analyzed by methods well known to those of skill in the art such as, for example, single crystal X-ray diffraction, X-ray powder diffraction, polarizing optical microscopy, thermal microscopy, thermogravimetry, differential thermal analysis, differential scanning calorimetry, IR spectroscopy, Raman spectroscopy and NMR spectroscopy. (Brittain, H., Chapter 6, pp. 205-208 in Polymorphism in Pharmaceutical Solids, (Brittain, H. ed.), Marcel Dekker, Inc. New York, (1999)). In addition, many commercial companies routine offer services that include preparation and/or characterization of solvates such as, for example, HOLODIAG, Pharmaparc II, Voie de l'Innovation, 27 100 Val de Reuil, France (http://www.holodiag.com).

“Subject,” “individual” or “patient” is used interchangeably herein and refers to a vertebrate, preferably a mammal. Mammals include, but are not limited to, murines, rodents, simians, humans, farm animals, sport animals and pets.

“Treating” or “treatment” of any disease or disorder refers, in some embodiments, to ameliorating the disease or disorder (i.e., arresting or reducing the development of the disease or at least one of the clinical symptoms thereof). Treatment may also be considered to include preemptive or prophylactic administration to ameliorate, arrest or prevent the development of the disease or at least one of the clinical symptoms. Treatment can also refer to the lessening of the severity and/or the duration of one or more symptoms of a disease or disorder. In a further feature, the treatment rendered has lower potential for long term side effects over multiple years. In other embodiments “treating” or “treatment” refers to ameliorating at least one physical parameter, which may not be discernible by the patient. In yet other embodiments, “treating” or “treatment” refers to inhibiting the disease or disorder, either physically, (e.g., stabilization of a discernible symptom), physiologically, (e.g., stabilization of a physical parameter) or both. In yet other embodiments, “treating” or “treatment” refers to delaying the onset of the disease or disorder.

“Therapeutically effective amount” means the amount of a compound that, when administered to a patient for treating a disease, is sufficient to effect such treatment for the disease. The “therapeutically effective amount” will vary depending on the compound, the disease and its severity and the age, weight, adsorption, distribution, metabolism and excretion etc., of the patient to be treated.

“Vehicle” refers to a diluent, excipient or carrier with which a compound is administered to a subject. In some embodiments, the vehicle is pharmaceutically acceptable.

Described herein are methods of treating and/or preventing nausea and/or emesis in a subject, by administering 5HT₃ antagonists or pharmaceutical composition thereof via oral and/or nasal inhalation. For example, nausea and/or emesis caused by neoplastic disease, radiation sickness morning sickness during pregnancy, migraine, the side effects of operative procedures, cytological drugs used to treat cancer, opioids and general anesthetics may be treated and/or prevented by the methods described herein.

The methods, devices, and/or kits described herein may provide for fast, efficient treatment and/or prevention of nausea and/or emesis including acute and/or delayed nausea and acute and/or delayed emesis. For example, a subject suffering from acute nausea associated with a clinical treatment for cancer by radiation or cytotoxic agents may self-administer a 5HT₃ antagonist composition, via oral and/or nasal inhalation prior to, or several hours or several days after discharge from the clinic to treat the acute nausea. Such treatment is non-invasive and simple to self-administer (i.e., because administration by injection is not required) and the therapeutic onset of action is rapid. If a recurrence of nausea occurs, an additional dose can be readily administered without clinical intervention to administer the 5HT₃ antagonist composition intravenously or taking an oral dosage form which might not be tolerated due to the nausea. The rapid therapeutic effect and ease of administration via oral and/or nasal inhalation may enable fast relief, without deleterious side effects.

In a first aspect, methods of treating and/or preventing nausea and/or emesis including acute and/or delayed nausea and/or acute and/or delayed emesis in a subject are provided. The methods include administering to the subject in need thereof, a therapeutically effective amount of a 5HT₃ antagonist or a pharmaceutical composition thereof by nasal inhalation and/or oral inhalation prior to an event which induces acute and/or delayed nausea and/or acute and/or delayed emesis. In some embodiments, the event is administration of chemotherapy, radiation or anesthesia.

In some embodiments, the 5HT₃ antagonist or a pharmaceutical composition thereof is self-administered by the subject. In other embodiments, the 5HT₃ antagonist or a pharmaceutical composition thereof is self-administered by the subject without clinical supervision.

In some embodiments, the 5HT₃ antagonist or a pharmaceutical composition thereof is administered about thirty minutes prior to the event. In other embodiments, the 5HT₃ antagonist or a pharmaceutical composition thereof is administered about four hours prior to the event. In still other embodiments, the 5HT₃ antagonist or a pharmaceutical composition thereof is administered about eight hours prior to the event. In still other embodiments, the 5HT₃ antagonist or a pharmaceutical composition thereof is administered about twelve hours prior to the event. In still other embodiments, the 5HT₃ antagonist or a pharmaceutical composition thereof is administered about twenty-four hours prior to the event.

In some embodiments, a second therapeutically effective amount of a 5HT₃ antagonist or a pharmaceutical composition thereof is administered after the event which induces acute and/or delayed nausea and/or acute and/or delayed emesis. In other embodiments, the 5HT₃ antagonist or a pharmaceutical composition thereof is administered about twelve hours after the event. In still other embodiments, the 5HT₃ antagonist or a pharmaceutical composition thereof is administered about twenty-four hours after the event. In still other embodiments, the 5HT₃ antagonist or a pharmaceutical composition thereof is administered about forty-eight hours after the event. In still other embodiments, the 5HT₃ antagonist or a pharmaceutical composition thereof is administered about ninety-six hours after the event. In still other embodiments, the 5HT₃ antagonist or a pharmaceutical composition thereof is administered about one hundred and twenty hours after the event. In some embodiments, additional therapeutically effective amounts of a 5HT₃ antagonist or a pharmaceutical composition thereof are administered to treat and/or prevent recurrence of nausea and/or emesis including acute and/or delayed nausea and/or acute and/or delayed emesis.

In a second aspect, methods of treating nausea and/or emesis including acute and/or delayed nausea and/or acute and/or delayed emesis in a subject are provided. The methods include administering to the subject in need thereof a therapeutically effective amount of a 5HT₃ antagonist or a pharmaceutical composition thereof, by nasal inhalation or oral inhalation. In some embodiments, the acute nausea and/or acute emesis is treated in less than about thirty minutes. In other embodiments, the acute nausea and/or acute emesis is treated in less than about fifteen minutes. In still other embodiments, the acute nausea and/or acute emesis is treated in less than about ten minutes.

In a third aspect, methods of preventing nausea and/or emesis including acute and/or delayed nausea and/or acute and/or delayed emesis in a subject are provided. The methods include administering to the subject in need thereof a therapeutically effective amount of a 5HT₃ antagonist or a pharmaceutical composition thereof, by nasal inhalation or oral inhalation. In some embodiments, the acute and delayed nausea and/or acute and delayed emesis is prevented for about seven days. In other embodiments, the acute and delayed nausea and/or acute and delayed emesis is prevented for about five days. In still other embodiments, the acute and delayed nausea and/or acute and delayed emesis is prevented for about three days. In still other embodiments, the acute and delayed nausea and/or acute and delayed emesis is prevented for about two days.

In some embodiments of the three aspects above, nausea and/or emesis is caused with neoplastic disease, radiation sickness, cytotoxic drugs, pregnancy, migraine, opioids or general anesthetics. In other embodiments of the three aspects above the 5HT₃ antagonist is not heated prior to inhalation.

In some embodiments of the three aspects above, the 5HT₃ antagonist is palonosetron, tropisetron, dolosetron, granisetron, ondansetron, metoclopramide, pancopride, zacopride, bemesetron, ricasetron, azesetron, cilansetron, alosetron, itasetron, zatosetron, lurosetron, lerisetron, ramosetron, mirisetron, indisetron or galdansetron. In other embodiments of the three aspects above, the 5HT₃ antagonist is palonosetron. In still other embodiments of the three aspects above, the 5HT₃ antagonist is pancopride. In still other embodiments of the three aspects above, the 5HT₃ antagonist is zacopride. In still other embodiments of the three aspects above, the 5HT₃ antagonist is tropisetron.

Inhalation devices may be of various designs, so long as can generate an aerosol of a 5HT₃ antagonist or pharmaceutical composition thereof. The devices generally include a housing having a proximal end and a body portion. A mouthpiece or nosepiece will typically be positioned at the proximal end. In some embodiments, the device may be a dry powder inhaler (DPI) with the composition adjusted to generate a significant portion of the delivered dose in the respirable range (drug particles less than approximately 5 microns median aerodynamic diameter (MMAD)). In other embodiments, the inhalation device may be a pressurized metered dose inhaler (pMDI) with the composition adjusted to generate a significant portion of the delivered dose in the respirable range (free drug or drug contained in propellant droplets having sizes less than approximately 5 microns median aerodynamic diameter (MMAD)). In still other embodiments, variations the pMDI or DPI can be fitted with nosepiece adapters to administer the drug laden dry powder or propellant to the nasopharynx. In still other embodiments, the pMDI and or DPI can be conventionally fitted with a mouthpiece, but the compositions may be adjusted to generate a significant portion of the delivered dose in the nonrespirable range (free drug particles or drug contained in propellant droplets greater than approximately 10 microns median aerodynamic diameter (MMAD)) so that most of the drug is deposited in the oropharynx.

In a DPI, the dosage is stored in the form of a non-pressurized dry powder and on actuation of the inhaler, the particles of the powder are inhaled by the subject. Similar to pMDIs, a compressed gas may be used to dispense the powder. Alternatively, when the DPI is breath-actuated, the powder may be packaged in various forms, such as a loose powder, cake or pressed shape in a reservoir. Examples of these types of DPIs include the Turbohaler™ inhaler (Astrazeneca, Wilmington, Del.) and Clickhaler® inhaler (Innovata, Ruddington, Nottingham, UK). When a doctor blade or shutter slides across the powder, cake or shape, the powder is culled into a flowpath whereby the patient can inhale the powder in a single breath. Other powders are packaged as blisters, gel caps, tabules, or other preformed vessels that may be pierced, crushed, or otherwise unsealed to release the powder into a flowpath for subsequent inhalation. Typical of these are the Diskus™ inhaler (Glaxo, Greenford, Middlesex, UK), EasyHaler® (Orion, Expoo, FI), and Novohaler™, Handihaler® (Boerhinger-Ingelheim) inhalers. Still other inhalers release the powder into a chamber or capsule and use mechanical or electrical agitators to keep the drug suspended for a short period until the patient inhales. Examples of this are the Exubera® inhaler (Pfizer, New York, N.Y.), Qdose inhaler (Microdose, Monmouth Junction, N.J.), and Spiros® inhaler (Dura, San Diego, Calif.).

pMDIs generally have two components: a canister in which the drug particles are stored under pressure in a suspension or solution form, and a receptacle used to hold and actuate the canister. The canister may contain multiple doses of the composition, although it is possible to have single dose canisters as well. The canister may include a valve, typically a metering valve, from which the contents of the canister may be discharged. Aerosolized drug is dispensed from the pMDI by applying a force on the canister to push it into the receptacle, thereby opening the valve and causing the drug particles to be conveyed from the valve through the receptacle outlet. Upon discharge from the canister, the drug particles are atomized, forming an aerosol. pMDIs generally use propellants to pressurize the contents of the canister and to propel the drug particles out of the receptacle outlet. In pMDIs, the composition is provided in liquid form and resides within the canister along with the propellant. The propellant may take a variety of forms. For example, the propellant may be a compressed gas or a liquefied gas. Chlorofluorocarbons (CFC) were once commonly used as liquid propellants, but have now been banned. They have been replaced by the now widely accepted hydrofluoroalkane (HFA) propellants.

In some embodiments, a manual discharge of aerosolized drug must be coordinated with inhalation, so that the drug particles are entrained within the inspiratory air flow and conveyed to the lungs. In other embodiments, a breath-actuated trigger, such as that included in the Tempo® inhaler (Allergan, Irvine Calif.) may be employed, which simultaneously discharges a dose of drug upon sensing inhalation (i.e., the device automatically discharges the drug aerosol when the user begins to inhale).

Nebulizers are liquid aerosol generators that convert bulk liquids, usually aqueous-based compositions, into mists or clouds of small droplets, having diameters less than 5 microns mass median aerodynamic diameter (MMAD), which can be inhaled into the lower respiratory tract. The bulk liquid contains particles of the therapeutic agent(s) or a solution of the therapeutic agent(s) and any necessary excipients. The droplets carry the therapeutic agent(s) into the nose, upper airways or deep lungs when the aerosol cloud is inhaled.

Pneumatic (jet) nebulizers use a pressurized gas supply as a driving force for liquid atomization. Compressed gas is delivered through a nozzle or jet to create a low pressure field which entrains a surrounding bulk liquid and shears it into a thin film or filaments. The film or filaments are unstable and break up into small droplets which are carried by the compressed gas flow into the inspiratory breath. Baffles inserted into the droplet plume screen out the larger droplets and return them to the bulk liquid reservoir. Examples include the PARI LC® Plus®, or Sprint® nebulizers, the Devilbiss PulmoAide® nebulizer and the Boehringer Ingelheim Respimat® inhaler.

Electromechanical nebulizers use electrically generated mechanical force to atomize liquids. The electromechanical driving force is applied by vibrating the bulk liquid at ultrasonic frequencies or by forcing the bulk liquid through small holes in a thin film. The forces generate thin liquid films or filament streams which break up into small droplets to form a slow moving aerosol stream which can be entrained in a respiratory flow.

One form of electromechanical nebulizers is ultrasonic nebulizers, in which the bulk liquid is coupled to a vibrator oscillating at frequencies in the ultrasonic range. The coupling is achieved by placing the liquid in direct contact with the vibrator such as a plate or ring in a holding cup, or by placing large droplets on a solid vibrating projector. The vibrations generate circular standing films which break up into droplets at their edges to atomize the liquid. Examples include the DuroMist® nebulizer, Drive Medical's Beetle Neb® nebulizer, Octive Tech's Densylogic® nebulizer and the John Bunn Nano-Sonic® nebulizer.

Another form of an electromechanical nebulizer is a mesh nebulizer, in which the bulk liquid is driven through a mesh or membrane with small holes ranging from 2 to 8 microns in diameter, to generate thin filaments which immediately break up into small droplets. In certain designs, the liquid is forced through the mesh by applying pressure with a solenoid piston driver (AERx®) or by sandwiching the liquid between a piezoelectrically vibrated plate and the mesh, which results in an oscillatory pumping action (EFlow®, AerovectRx, TouchSpray™). In a second design type the mesh vibrates back and forth through a standing column of the liquid to pump it through the holes. Examples include the AeroNeb®, AeroNeb Go®, Pro®; PARI EFlow®; Omron 22UE®; and Aradigm AERx®.

In some embodiments, a combination of compounds may be used in conjunction with one or more 5HT₃ antagonists, either in the same composition, or in different composition. The different compounds may be administered at the same time or at different times. In other embodiments, one or more 5HT₃ antagonists may be combined with one or more dopamine antagonists such as, for example, metoclopramide, domperidone, chlorproperazine, promethazine, thiethylperazine, droperidol, prochorperazine, scopolamine or combinations thereof. In still other embodiments, one or more 5HT₃ antagonists may be combined with one or more with one or more sedatives or sedative-hypnotics, such as, for example, alprazolam (e.g., Xanax®), clonazepam (e.g., Klonopin®), diazepam, temazepam (e.g., Restoril®), flunitrazepam (e.g., Rohypnol®), triazolam (e.g., Halcion®), flurazepam (e.g., Dalmane®), nitrazepam (e.g., Mogadon®), midazolam (e.g., Versed®) or combinations thereof. In still other embodiments, one or more 5HT₃ antagonists may be combined with one or more with one or more glucocorticosteroids, such as, for example, budesonide, fluticasone, triamcinolone, beclomethasone, ciclesonide, flunisolide, dexamethasone, prednisolone, prednisone or combinations thereof. In still other embodiments, one or more 5HT₃ antagonists may be combined with one or more with one or more NK1 antagonists, such as, for example, aprepitant, netupitant, fosaprepitant, lanepitant, casopitant, dapitant, exlopitant, befetupitant, burapitant, vepitant, rolapitant, serlopitant, telmapitant, vofopitant, maropitant or combinations thereof. In still other embodiments, one or more 5HT₃ antagonists may be combined with one or more with one or more cannabinoids, such as for example, tetrahydocannibinol, cannibinol, cannibigerol, tetrahydrocannabivarin, cannibidivarin, cannibichromene or combinations thereof. In still other embodiments, one or more 5HT₃ antagonists may be combined with one or more with one or more nonclassical cannabinoids, such as for example, aminoalkylindoles, 1,5-diarylpyrazoles, quinolines, aryl sulfonamides or combinations thereof. In still other embodiments, one or more 5HT₃ antagonists may be combined with one or more with one or more endocannabinoids, such as for example, arachidonoylethanolamine, 2-arachidonoylglycerol, N-arachidonoyl dopamine, virodhamine lysophosphatidylinositol or combinations thereof. In still other embodiments, one or more 5HT₃ antagonists may be combined with one or more with one or more antihistamines such as, for example, dimenhydrinate, dipheyhydramine, meclozine or combinations thereof. It should be understood that one or more 5HT₃ antagonists can be combined with one or more of any of the therapeutic agents above.

Pharmaceutical compositions may include one or more 5HT₃ antagonists in any appropriate amount. For example, a pharmaceutical composition may include a 5HT₃ antagonist in an amount of from about 1% to about 99% by weight of the composition. In some embodiments, the 5HT₃ antagonist may be included in an amount of from about 0.05% to about 10% by weight of the composition. It is understood that the above dosages are exemplary, and that there may be instances in which higher or lower dosages may be merited.

In some embodiments, the amount of the 5HT₃ antagonist may be selected to achieve a certain plasma concentration by, for example, aerosol administration (e.g., using pMDI). As an example, the dose range for the 5HT₃ antagonist may be from about 0.5 μg/kg to about 500 μg/kg (e.g., from about 1.0 μg/kg to about 150 μg/kg, from about 2.0 μg/kg to about 50.0 μg/kg, from about 2.5 μg/kg to about 25.0 μg/kg, from about 3.0 μg/kg to about 10.0 μg/kg, from about 3.0 μg/kg to about 5.0 μg/kg)., to be administered in about 1 minute or slower. The corresponding desired plasma concentration may range between about 0.0015 ng/ml and about 600 ng/ml, depending on the general condition of the subject.

In addition to including the 5HT₃ antagonist, the pharmaceutical compositions may further include additional ingredients, such as preservatives, buffers, antioxidants and stabilizers, nonionic wetting or clarifying agents, viscosity-increasing agents, absorption-enhancing agents, pH modifying agents and co-solvents and the like.

For example, inhalation aerosols from dry powder inhalers, nebulizers, vaporizers and pressurized metered dose inhalers typically include excipients or solvents to increase stability or deliverability of these drugs in an aerosol form. As an example, nebulizers generate an aerosol from a liquid, some by breakup of a liquid jet and some by ultrasonic vibration of the liquid with or without a nozzle.

Liquid compositions are prepared and stored under aseptic or sterile conditions to prevent microorganism growth and thus the use of preservatives is contemplated. Additionally, solvents, detergents and other agents may be used to stabilize the drug composition. As another example, for pMDI's, the compositions may be formulated in a canister under pressure with a solvent and propellant mixture, such as, for example, chlorofluorocarbons or hydrofluoroalkanes. Upon being dispensed, a jet of the mixture is ejected through a valve and nozzle and the propellant “flashes off”, leaving an aerosol of the compound.

Examples of absorption-enhancing agents include, for example, N-acetylcysteine, polyethylene glycols, caffeine, cyclodextrins, glycerol, alkyl saccharides, lipids, lecithin, dimethylsulfoxide, and the like. Examples of preservatives for use in a solution include, for example, polyquaternium-1, benzalkonium chloride, thimerosal, chlorobutanol, methyl paraben, propyl paraben, phenylethyl alcohol, disodium edetate, sorbic acid, benzethonium chloride, and the like. Typically, (but not necessarily) such preservatives may be employed at a level from about 0.001% to about 1.0% by weight. Examples of buffers include, for example, citric acid, sodium and potassium bicarbonates, sodium and potassium borates, sodium and potassium carbonates, sodium acetate, sodium biphosphate and the like. In some embodiments, the buffers may be included in amounts sufficient to maintain the pH of the composition at between about pH 3 and about pH 9 (e.g., between about pH 4 and about pH 7.5).

Suitable antioxidants and stabilizers include, for example, ascorbic acid, sodium bisulfite, sodium metabisulfite, sodium thiosulfite, thiourea, caffeine, chromoglycate salts, cyclodextrins and the like. Suitable wetting and clarifying agents include, for example, polysorbate 80, polysorbate 20, oleic acid, lecithin and other phospholipids, poloxamer 282 and tyloxapol. Suitable viscosity-increasing agents include, for example, dextran 40, dextran 70, gelatin, glycerin, hydroxyethylcellulose, hydroxmethylpropylcellulose, lanolin, methylcellulose, petrolatum, polyethylene glycol, polyvinyl alcohol, polyvinylpyrrolidone, carboxymethylcellulose and the like.

The particle size of aerosols may be controlled to provide desired characteristics. For example, when using DPIs, the drug particles may be generated from the bulk drug by attrition processes (e.g., grinding, micronizing, milling, etc.), or by multiphase precipitation processes (e.g., spray drying, solution precipitation, supercritical extraction/precipitation, lyophilization, etc.) to yield powders that may be dispersed in a propellant to obtain an acceptable particle size for delivery to the lungs. As dry powder compositions are prone to aggregation and low flowability, which may result in diminished efficiency, scrupulous attention may be required during milling, blending, powder flow, filling and even administration to ensure that the dry powder aerosols are reliably delivered and have the proper particle size distribution for efficacious delivery to the lungs. In some embodiments, the particles comprising the aerosol have a median aerodynamic diameter ranging from about 1 μM to about 10 μM. In other embodiments, particles comprising the aerosol have a median aerodynamic diameter ranging from about 1 μM to about 5 μM. In still other embodiments, particles comprising the aerosol have a median aerodynamic diameter ranging from about 1 μM to about 3 μM.

In some embodiments, the 5HT₃ antagonists or pharmaceutical compositions thereof described herein are administered by inhalation using devices such as pressurized metered dose inhalers, breath-actuated inhalers or dry powder inhalers. In general, a subject will first completely exhale. Then the subject inhales through the mouthpiece, establishing air flow through the device. For manually actuated devices, the subject must actuate the discharge of drug aerosol as upon inhalation. For breath-actuated devices, the device automatically discharges the drug aerosol when the subject begins to inhale. The subject continues to inhale to fill the lungs to capacity and then may hold breath for a period of time to allow the aerosolized drug to settle within the airways deep in the lungs.

The 5HT₃ antagonists or pharmaceutical compositions thereof may be administered at a frequency selected to treat the particular disorder. For example, the 5HT₃ antagonists or pharmaceutical compositions thereof may be administered once a day, several times a day, weekly or monthly. The 5HT₃ antagonists or pharmaceutical compositions thereof may be administered via a single dosage, two dosages or multiple dosages. As an example, a subject suffering from nausea may administer the 5HT₃ antagonists or pharmaceutical compositions thereof by a single administration or a couple of administrations and then may not administer the antagonists or pharmaceutical compositions thereof again, until nausea reoccurs. In some embodiments, the 5HT₃ antagonist or a pharmaceutical composition thereof is self-administered by the subject. In other embodiments, the 5HT₃ antagonist or a pharmaceutical composition thereof is self-administered by the subject without clinical supervision.

In some embodiments, when a 5HT₃ antagonist or pharmaceutical composition thereof is administered with another therapeutic agent, the other therapeutic agent is administered prior to administration of the 5HT₃ antagonist or pharmaceutical composition thereof. In other embodiments, when a 5HT₃ antagonist or pharmaceutical composition thereof is administered with another therapeutic agent, the other therapeutic agent is co-administered with the 5HT₃ antagonist or pharmaceutical composition thereof. In still other embodiments, when a 5HT₃ antagonist or pharmaceutical composition thereof is administered with another therapeutic agent, the other therapeutic agent is administered after administration of the 5HT₃ antagonist or pharmaceutical composition thereof.

In some embodiments, when a 5HT₃ antagonist or pharmaceutical composition thereof is administered with another therapeutic agent, the other therapeutic agent is administered by oral and/or nasal inhalation. In other embodiments, when a 5HT₃ antagonist or pharmaceutical composition thereof is administered with another therapeutic agent, the other therapeutic agent is administered by oral, nasal, transdermal, transmucosal, rectal, buccal or other any other non-invasive route by methods well know to the skilled artisan.

Oral dosage forms are either solid, gel or liquid. The solid dosage forms are tablets, capsules, granules and bulk powders. Types of oral tablets include compressed, chewable lozenges and tablets which may be enteric-coated, sugar-coated or film-coated. Capsules may be hard or soft gelatin capsules, while granules and powders may be provided in non-effervescent or effervescent form with the combination of other ingredients known to those skilled in the art.

In certain embodiments, the formulations are solid dosage forms such as, for example, capsules or tablets. The tablets, pills, capsules, troches and the like may contain one or more of the following ingredients, or compounds of a similar nature: a binder; a lubricant; a diluent; a glidant; a disintegrating agent; a coloring agent; a sweetening agent; a flavoring agent; a wetting agent; an enteric coating; a film coating agent and modified release agent. Examples of binders include microcrystalline cellulose, methyl paraben, polyalkyleneoxides, gum tragacanth, glucose solution, acacia mucilage, gelatin solution, molasses, polyvinylpyrrolidine, povidone, crospovidones, sucrose and starch and starch derivatives. Lubricants include talc, starch, magnesium/calcium stearate, lycopodium and stearic acid. Diluents include, for example, lactose, sucrose, trehalose, lysine, leucine, lecithin, starch, kaolin, salt, mannitol and dicalcium phosphate. Glidants include, but are not limited to, colloidal silicon dioxide. Disintegrating agents include crosscarmellose sodium, sodium starch glycolate, alginic acid, corn starch, potato starch, bentonite, methylcellulose, agar and carboxymethylcellulose. Coloring agents include, for example, any of the approved certified water soluble FD and C dyes, mixtures thereof; and water insoluble FD and C dyes suspended on alumina hydrate and advanced coloring or anti-forgery color/opalescent additives known to those skilled in the art. Sweetening agents include sucrose, lactose, mannitol and artificial sweetening agents such as saccharin, and any number of spray dried flavors. Flavoring agents include natural flavors extracted from plants such as fruits and synthetic blends of compounds which produce a pleasant sensation or mask unpleasant taste, such as, but not limited to peppermint and methyl salicylate. Wetting agents include propylene glycol monostearate, sorbitan monooleate, diethylene glycol monolaurate and polyoxyethylene lauryl ether. Enteric-coatings include fatty acids, fats, waxes, shellac, ammoniated shellac and cellulose acetate phthalates. Film coatings include hydroxyethylcellulose, sodium carboxymethylcellulose, polyethylene glycol 4000 and cellulose acetate phthalate. Modified release agents include polymers such as the Eudragit® series and cellulose esters.

Liquid oral dosage forms include aqueous solutions, emulsions, suspensions, solutions and/or suspensions reconstituted from non-effervescent granules and effervescent preparations reconstituted from effervescent granules. Aqueous solutions include, for example, elixirs and syrups. Emulsions are either oil-in-water or water-in-oil.

Topical mixtures may be a solution, suspension, emulsions or the like and are formulated as creams, gels, ointments, emulsions, solutions, elixirs, lotions, suspensions, tinctures, pastes, foams, aerosols, irrigations, sprays, suppositories, bandages, dermal patches or any other formulations suitable for topical administration. Topical administration is contemplated for transdermal delivery and also for administration to the eyes or mucosa, or for inhalation therapies. Nasal solutions of the other therapeutic agent alone or in combination with other excipients can also be administered. Solutions, particularly those intended for ophthalmic use, may be formulated as 0.01%-10% isotonic solutions, pH about 5-7.4, with appropriate salts. Other routes of administration, such as transdermal patches, including iontophoretic and electrophoretic devices and rectal administration are also contemplated herein. Transdermal patches, including iontophoretic and electrophoretic devices, are well known to those of skill in the art. For example, such patches are disclosed in U.S. Pat. Nos. 6,267,983, 6,261,595, 6,256,533, 6,167,301, 6,024,975, 6,010715, 5,985,317, 5,983,134, 5,948,433 and 5,860,957.

Dosage forms for rectal administration include, for example, rectal suppositories, capsules and tablets for systemic effect. Rectal suppositories, as used herein, include solid bodies for insertion into the rectum which melt or soften at body temperature releasing one or more pharmacologically or therapeutically active ingredients. Substances utilized in rectal suppositories are bases or vehicles and agents to raise the melting point. Examples of bases include cocoa butter (theobroma oil), glycerin-gelatin, carbowax (polyoxyethylene glycol) and appropriate mixtures of mono-, di- and triglycerides of fatty acids. Combinations of the various bases may be used. Agents to raise the melting point of suppositories include spermaceti and wax. Rectal suppositories may be prepared either by compression or by molding. The weight of a rectal suppository, in some embodiments, is about 2 to 3 gm. Tablets and capsules for rectal administration are manufactured using the same substance and by the same methods as for formulations for oral administration.

The dosing regimen employed may depend on a number of factors, including the cause of nausea and/or emesis, the severity of the symptoms, and the purpose of the treatment or prevention regimen.

The 5HT₃ antagonist or pharmaceutical composition thereof may be administered in any appropriate dose. In some embodiments, the dose may be from 0.5 μg/kg to about 500 μg/kg (e.g., from about 1.0 μg/kg to about 150 μg/kg, from about 2.0 μg/kg to about 50.0 μg/kg, from about 2.5 μg/kg to about 25.0 μg/kg, from about 3.0 μg/kg to about 10.0 μg/kg, from about 3.5 μg/kg to about 5.0 μg/kg). In some embodiments, the dose may range from between about 75 μg to about 300 μg and multiple doses may be administered. However, the above is exemplary and lower or higher dosing regimens that differ up to 5% or 10% in range may be required.

In some embodiments, treatment may be achieved in about 30 minutes or less (e.g., about 15 minutes or less, about 10 minutes or less, about 5 minutes or less). In other embodiments, treatment may be achieved within a range from about 0.5 minutes to about 30 minutes, about 1 minute to about 15 minutes, about 1 minute to about 10 minutes and about 1 minute to about 5 minutes.

In some embodiments, the time following administration of the 5HT₃ antagonist or pharmaceutical composition thereof, at which the peak plasma concentration is attained (Tₘₐₓ) may be about 30 minutes or less (e.g., about 20 minutes or less, about 15 minutes or less, about 10 minutes or less or about 5 minutes or less). In certain embodiments, the plasma concentration of the 5HT₃ antagonist or pharmaceutical composition thereof in the subject at about 15 minutes or less after administration may be from about 0.0015 ng/mL to about 600 ng/mL.

In some embodiments, the plasma concentration of the 5HT₃ antagonist or pharmaceutical composition thereof is at a therapeutically effective concentration of about fifteen minutes after administration, and remains at a therapeutically effective concentration of between 0.5 and 10 ng/mL for up to 168 hours. In other embodiments, the therapeutically effective concentration is between about 0.1 ng/mL and about 100 ng/mL within about fifteen minutes. In still other embodiments, the therapeutically effective concentration remains at a between about 0.5 ng/mL and about 10 ng/mL for up to about one hundred and sixty-eight hours.

The devices and/or the 5HT₃ antagonist or pharmaceutical composition thereof described here may be packaged and/or distributed (e.g., to hospitals, clinics, physicians, and/or patients) in an administration kit. Such kits may comprise one or more inhalation devices and one or more containers (e.g., unit doses or multi-dose containers) of the 5HT₃ antagonist or pharmaceutical composition thereof. In some embodiments, the kit may include one or more devices that are already loaded with the 5HT₃ antagonist or pharmaceutical composition thereof. For example, a device may comprise a reservoir that is pre-filled with the 5HT₃ antagonist or pharmaceutical composition thereof. In other embodiments, kits may include multiple different 5HT₃ antagonists or pharmaceutical compositions thereof, and/or multiple different dosages of the same 5HT₃ antagonist or pharmaceutical composition thereof. In still other embodiments, kits may additionally include a carrier or diluent, a case, instructions for operating the appropriate device, instructions for administering 5HT₃ antagonist or pharmaceutical composition thereof or combinations thereof.

### EXAMPLES

The following examples are intended to be illustrative and not to be limiting.

### Example 1

For pressurized metered dose inhalation, coated 10 ml MDI cans (Presspart Ltd, UK) are crimped with BK357 50 mcl valves (Bespak Ltd, UK), and a suspension (about 38 mg palonosetron chloride in about 7.5 mL HFA 135a) is filled through the valve.

### Example 2

For pressurized metered dose inhalation, standard 10 ml MDI cans (Presspart Ltd, UK) are crimped with BK357, 50 mcl valves (Bespak Ltd, UK), and a suspension (about 75 mg palonosetron citrate in about 7.5 mL HFA 227) is filled through the valve. If tested at 28.3 LPM through an Andersen Cascade Impactor in a BK636 actuator (Bespak Ltd, UK), the fine particle fraction of palonosetron citrate <4.7 microns is anticipated to be >25%.

### Example 3

For pressurized metered dose inhalation, coated 10 ml MDI cans (Presspart Ltd, UK) are crimped with BK357, 50 mcl valves (Bespak Ltd, UK), and a suspension (about 38 mg palonosetron chloride in about 7.5 mL HFA 134a:227 at a ratio of 30:70 v/v) is filled through the valve containing 0.1% inhalation grade oleic acid (Super Refined Grade, Croda, UK). If tested at 28.3 LPM through an Andersen Cascade Impactor in a BK636 actuator (Bespak Ltd, UK), the fine particle fraction of palonosetron chloride <4.7 microns is anticipated to be >25%.

### Example 4

For dry powder inhalation, 30 mg portions of an 8% (w/w) blended composition consisting of palonosetron chloride having particle sizes between 1.0 and 4.5 microns MMAD, and a lactose carrier composition is provided. The lactose blend composition is a blend of two carriers, Respitose® SV003, present at 95% (w/w) with particle sizes of between about 30 about 100 microns, and Respitose® LH300 (Lactohale 300) present at 5% (w/w) with particle sizes less than 10 microns which are filled into size #3 HPMC capsules and single capsules were placed into an Plastiape® RS00 Model 8 dry powder inhaler. If tested at 60 LPM through a Next Generation Impactor, the fine particle fraction of palonosetron chloride <4.7 microns is anticipated to be >25%. A range 0.5 to 20% palonosetron is practicable.

### Example 5

For dry powder inhalation, 25 mg portions of a 5% (w/w) blended composition consisting of palonosetron chloride of particle sizes between 1 and 4 microns MMAD, and lactose (Pharmatose® 200M, DMV, Netherlands) are filled into size #3 HPMC capsules and single capsules are placed into an Plastiape® RS00 Model 8 dry powder inhaler. If tested at 60 LPM through a Next Generation Impactor, the fine particle fraction of palonosetron chloride <4.7 microns is anticipated to be >25%. A range 0.5 to 20% palonosetron is practicable.

### Example 6

For a nebulizer solution for inhalation, a 0.125% w/w solution of palonosetron chloride is formulated in a pharmaceutically acceptable buffer for inhalation (0.85% sodium chloride, 0.062% sodium citrate, 0.019% citric acid in water). If tested through a Next Generation Impactor at 15 LPM, the fine particle fraction of palonosetron <4.7 microns is anticipated to be >20%.

While methods, devices, and kits have been described in some detail here by way of illustration and example, such illustration and example is for purposes of clarity of understanding only. It will be readily apparent to those of ordinary skill in the art in light of the teachings herein that certain changes and modifications may be made thereto without departing from the spirit and scope of the invention as recited in the appended claims.

1. A method of treating acute nausea and/or acute emesis in a subject comprising administering to the subject in need thereof a therapeutically effective amount of a 5HT₃ antagonist or a pharmaceutical composition thereof by nasal inhalation and/or oral inhalation.

2. A method of preventing acute and/or delayed nausea and/or acute and/or delayed emesis in a subject comprising administering to the subject in need thereof a therapeutically effective amount of a 5HT₃ antagonist or a pharmaceutical composition thereof, wherein the 5HT₃ antagonist is administered by nasal inhalation and/or oral inhalation.

3. A method of treating and/or preventing acute and/or delayed nausea and/or acute and/or delayed emesis in a subject comprising administering to the subject in need thereof a therapeutically effective amount of a 5HT₃ antagonist or a pharmaceutical composition thereof by nasal inhalation and/or oral inhalation prior to an event which induces acute and/or delayed nausea and/or acute and/or delayed emesis.

4. The method of claim 1 further comprising administering a second therapeutically effective amount of a 5HT₃ antagonist or a pharmaceutical composition thereof after the event which induces acute and/or delayed nausea and/or acute and/or delayed emesis.

5. The method of claim 1, wherein the 5HT₃ antagonist or a pharmaceutical composition thereof is administered about twelve hours after the event.

6. The method of claim 1, further comprising administering additional therapeutically effective amounts of a 5HT₃ antagonist or a pharmaceutical composition thereof to treat and/or prevent recurrence of acute and/or delayed nausea and/or acute and/or delayed emesis,

7. The method of claim 1, wherein nausea and/or emesis is associated with neoplastic disease, radiation sickness, cytotoxic drugs, pregnancy, migraine, opioids or general anesthetics.

8. The method of claim 1, wherein the 5HT₃ antagonist is palonosetron, tropisetron, dolosetron, granisetron, ondansetron, metoclopramide, pancopride, zacopride, bemesetron, ricasetron, azesetron, cilansetron, alosetron, itasetron, zatosetron, lurosetron, lerisetron, ramosetron, mirisetron, indisetron or galdansetron.

9. The method of claim 1, wherein the 5HT₃ antagonist is administered as an aerosol.

10. The method of claim 9, wherein the particles comprising the aerosol have an media aerodynamic diameter ranging from about 1 μM to about 10 μM.

11. The method of claim 1, wherein the 5HT₃ antagonist is administered by a device.

12. The method of claim 11, wherein the device is a nasal spray, a dry powder nasal delivery system, pressurized metered dose inhaler, a breath-actuated metered dose inhaler, a dry power inhaler or a nebulizer.

13. The method of claim 1, wherein the 5HT₃ antagonist is self-administered.

14. The method of claim 1, wherein Tₘₐₓ for administration of the 5HT₃ antagonist is less than about twenty minutes.

15. The method of claim 1, wherein the plasma concentration of the 5HT₃ antagonist at 15 minutes or less after administration is in the range of about 0.0015 ng/mL to about 600 ng/mL.

16. The method of claim 1 wherein the dosage of the 5HT₃ antagonist is between about 0.5 μg/kg to about 500 μg/, about 1.0 μg/kg to about 150 μg/kg, about 2.0 μg/kg to about 50.0 μg/kg, about 2.5 μg/kg to about 25.0 μg/kg, about 3.0 μg/kg to about 10.0 μg/kg, about 3.5 μg/kg to about 5.0 μg/kg.

17. The method of claim 1, wherein the 5HT₃ antagonist or pharmaceutical composition thereof is administered in multiple doses.

18. The method of claim 1, wherein the 5HT₃ antagonist or pharmaceutical composition thereof is administered in two doses.

19. The method of claim 1, further comprising administration of a therapeutically effective amount of a second therapeutic agent.

20. The method of claim 19, wherein the second therapeutic agent is co-administered with the 5HT₃ antagonist or pharmaceutical composition thereof
